(19) United States
(12) Patent Application Publication
(10) Pub. No.: US 2005/0003515 A1
(43) Pub. Date: Jan. 6, 2005

(54) Methane cleansing by microbial removal of H2S

(76) Inventor: Lewis J. Daly, Fayetteville, NY (US)

Correspondence Address: WALL MARJAMA & BILINSKI, 101 SOUTH SALINA STREET, SUITE 400, SYRACUSE, NY, 13202, US

(21) Appl. No.: 10/610,969

(22) Filed: Jul. 1, 2003

(51) Int. Cl. C12M 1/16; B01D 53/85

(52) U.S. Cl. 435/266; 435/299.1

(57) ABSTRACT

A system for removing H₂S from methane (CH₄) which includes providing at least one biofilter cartridge that functions to sustain microbial activity which will function to consume H₂S contained in a stream of methane gas. A stream of methane gas which contains H₂S is transported directly into a biofilter system which contains at least one cartridge containing selected microorganisms which function to biodegrade H₂S. Following treatment the methane gas is recirculated to a storage reservoir or a source of use.

### FIELD OF THE INVENTION

This invention relates generally to biofilteration, and more specifically to a system for the removal of hydrogen sulfide (H₂S) from gaseous fuels such as methane.

### BACKGROUND OF THE INVENTION

In the biofilteration industry typical problems which are focused on include odor control, VOC removal, and the treatment of petroleum hydrocarbons. A fourth problem which has yet to be successfully addressed in the field of biofilteration is the removal of noxious and deleterious sulfur from energy gases.

Organic material can be microbially digested in the absence of oxygen to form biogas containing mostly methane, carbon dioxide, and small amounts of hydrogen sulfide. Specific systems have been developed which allow dairy farmers to collect animal waste and turn it into biogas, which can then be used to generate useful heat and electricity.

These systems have been researched since the early 1970's but economic and operational barriers have curtailed their widespread implementation. Recent emphasis on environmental protection, renewable fuel sources, and distributed generation indicates that these systems will likely become more prevalent.

When methane containing H₂S is used as a fuel, such as to run a motor-generator, the combustion converts the H₂S to harmful sulfuric acid (H₂SO₄) which corrodes the generator and dramatically shortens the generator lifetime.

It can therefore be seen that there is a need in the field for a system which will reduce and/or eliminate H₂S from methane to provide a “cleaner” methane for use as a fuel.

### SUMMARY OF THE INVENTION

It is therefore an object of the present invention to overcome the problems of the prior art described above.

It is another object of the present invention to provide a method of H₂S removal from fuel gases.

It is another object of the present invention to provide a method of H₂S removal from organic gaseous fuel.

It is another object of the present invention to provide a method of H₂S removal from methane.

It is yet another object of the present invention to provide a biofilteration system for the removal of H₂S from fuel gases.

It is a further object of the present invention to provide a system for H₂S removal from fuel gases which utilizes a reactor cartridge.

The invention is directed to a system for removing H₂S from methane (CH₄) or other suitable gaseous fuel which includes providing at least one biofilter cartridge which sustains microbial activity that will function to consume H₂S contained in a stream of methane gas. In one embodiment, a stream of methane gas which contains H₂S is transported directly into a biofilter system which contains at least one cartridge containing selected microorganisms which function to biodegrade H₂S. The clean methane gas is then recirculated to a source of use.

A suitable reactor cartridge which can be used in the present invention includes an outer housing having a pair of oppositely disposed open ends. The housing which contains an inlet fan and an inlet port connected to a vertical inlet tube positioned at one end which also contains a water inlet and a gas outlet. A water reservoir is attached to the opposite end of the housing, with the reservoir further containing an outlet for the removal of water. In operation, a source of methane which contains H₂S to be treated is passed through the inlet and down the vertical tube and reverses direction at the bottom to go up through a suitable source of microorganisms contained in said housing which are selected to degrade the H₂S. The treated methane is released through said gas outlet, at the top of the housing and captured for storage or direct use.

### BRIEF DESCRIPTION OF THE DRAWINGS

For a further understanding of these and objects of the invention, reference will be made to the following detailed description of the invention which is to be read in connection with the accompanying drawing, wherein:

FIG. 1 is a side elevational view of a reactor cartridge used in the present invention having upward airflow.

FIG. 2 is a partial sectional view of the cartridge of FIG. 1 with the bottom of the cartridge disassembled.

FIG. 3 is a partial sectional view of the cartridge of FIG. 2 with the bottom assembled illustrating the down flow of the gas being treated opposite to FIG. 1.

FIG. 4 is a partial section view of the cartridge of FIG. 3 in which the inlet tube has been deleted.

### DETAILED DESCRIPTION OF THE INVENTION

In the present invention aerobic microorganisms are used to remove hydrogen sulfide from the gas stream and oxidize it back to sulfate, which then combines with water to form sulfuric acid.

Initial testing indicated that cow manure compost, which contains sulfur oxidizing bacteria, can remove hydrogens sulfide (H₂S) from a gas stream with removal efficiencies above 80%.

Farm digester gas is not the only source of sulfide contaminated methane rich gas for which biofilteration technology may be suitable. Wastewater treatment plants, landfills, paper mills, and food processing plants are all capable of producing biogas. Additionally, as higher quality natural gas wells are depleted, it may become economical to exploit smaller, remote, sulfur contaminated wells, biofilteration with its modular nature and low operating costs may be the ideal technology for sulfur removal from these gas source.

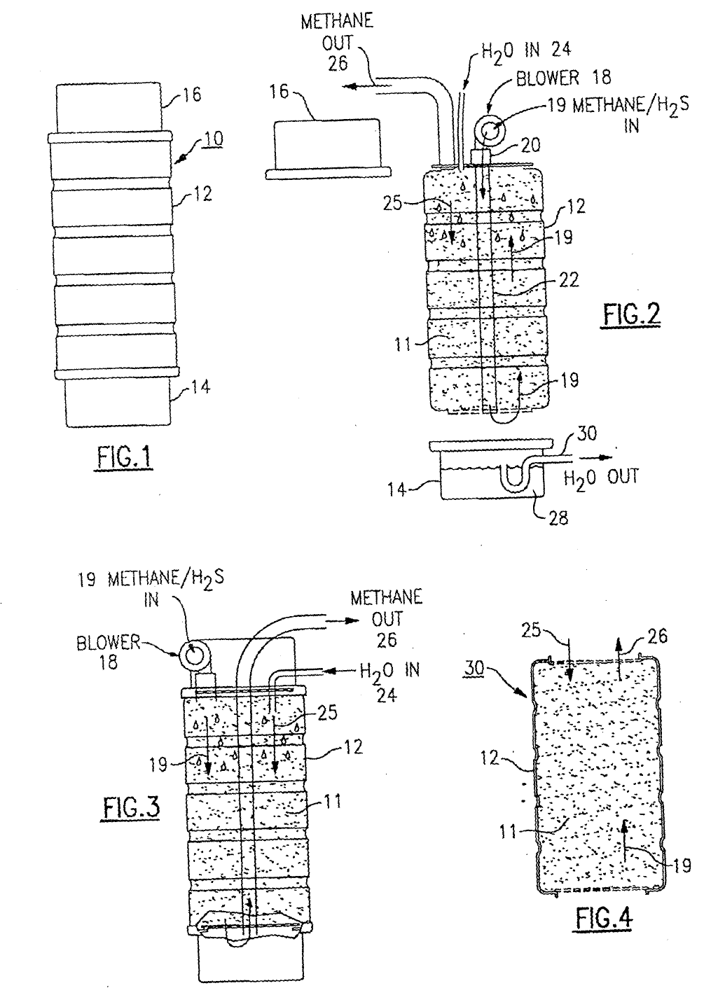

One embodiment exemplary of the invention is illustrated in FIGS. 1-3 is directed to a system for removing hydrogen sulfide (H₂S) from methane (CH₄) or other suitable gaseous fuel. As shown in FIGS. 1-3 at least one biofilter cartridge 10 which sustains microbial activity can be used to consume H₂S contained in a stream of methane gas 19 that is introduced to the microorganisms at the top or bottom of the carrier media 11. As shown by the arrows in the drawings, a stream of methane gas which contains H₂S is transported directly into a biofilter system which contains at least one cartridge containing selected microorganisms which function to biodegrade H₂S. The clean methane gas 26 is then recirculated to a source of use.

Carrier 11 may comprise any artificial or synthetic such as granular inert plastics, ceramics or crystalline materials which support and act as a carrier for the microorganisms or bacteria. Pearlite functions as an economical and readily available carrier or media. Any suitable microorganism or bacteria which functions to remove or biodegrade H₂S can be used. Suitable microorganism include Thiobacillus and Ralstonia. If a natural occurring or biodegradable material is used in place of a synthetic carrier, such as cow manure compost, tree bark or vegetation, then microorganisms indigenous to these materials will function to biodegrade the H₂S.

A suitable reactor cartridge 10 which can be used in the present invention includes an outer housing 12 having a pair of oppositely disposed open ends which are closed by a bottom 14 and a top 16. The housing contains an inlet fan or blower 18 and an inlet port 20 connected to a vertical inlet tube 22 positioned at one end which also contains a flush water inlet 24 and a gas outlet 26. A water reservoir 28 is contained in bottom member 14 at the opposite end of the housing, with the reservoir further containing an outlet 30 for the removal of flush water. In operation, a source of methane 19 which contains H₂S to be treated is passed through the inlet and down the vertical tube 22 and reverses direction at the bottom to go up through a suitable source of microorganisms contained in said housing which are selected to degrade the H₂S. The treated methane is released through said gas outlet, at the top of the housing and captured for storage or direct use. The remote cartridge is also flushed by the down flow of water 25.

FIG. 4 illustrates an alternative embodiment of the present invention in which inlet tube 22 has been removed, and the methane flow 19 is from the bottom upward through carrier 11.

While the present invention has been particularly shown and described with reference to the preferred mode as illustrated in the drawing, it will be understood by one skilled in the art that various changes in detail may be effected therein without departing from the spirit and scope of the invention as defined by the claims including applications and use for cleansing other gaseous fuels of unwanted secondary organic contaminants.

1. A system for removing H₂S from methane (CH₄) which includes providing at least one biofilter cartridge that functions to sustain microbial activity that will function to consume H₂S contained in a stream of methane gas which comprises establishing a stream of methane gas which contains H₂S and transporting said methane gas stream directly into a biofilter system which contains at least one cartridge containing selected microorganisms which function to biodegrade H₂S followed by recirculating the treated methane to a storage reservoir or a source of use.

2. The system of claim 1 in which the cartridge is positioned vertically and water is circulated through the microorganisms.

3. The system of claim 1 where water is periodically flushed through the microorganisms at a fast rate.

4. The system of claim 1 where said water is filtered, pH neutralized, and recirculated.

5. The system of claim 1 where said water is filtered and neutralized (pH).

6. The system of claim 1 in which the microorganisms are at least one isolated from the group consisting of bacteria.

7. The system of claim 1 in which the cartridges are positioned vertically and water is continuously trickled through the microorganisms.

8. The system of claim 2 in which the microorganism laden water film is supported on a carrier material.

9. The system of claim 7 in which the carrier is at least one artificial material selected from the group consisting of granular inert plastics.

10. The system of claim 7 in which the carrier is at least one artificial material selected from the group consisting of granular materials.

11. The system of claim 7 in which the carrier is at least one material selected from the group consisting of crystalline minerals.

12. The system of claim 7 in which the carrier or media consists of pearlite.

13. The system of claim 7 in which the carrier may include at least one natural material selected from a group consisting of biodegradable media such as compost, tree bark and vegetation.

14. A vertical reactor cartridge which comprises an outer housing having a pair of oppositely disposed open ends, a housing which contains an inlet fan and an inlet port connected to a vertical inlet tube positioned at one end extending through center with said end further containing a water inlet and a gas outlet, and a water reservoir attached to said opposite end, with said reservoir further containing an outlet for the removal of water, where in operation, a source of methane which contains H₂S to be treated is passed through said inlet and passed through a suitable source of microorganisms contained in said housing which are selected to degrade the H₂S, with said treated methane being released through said gas outlet.

15. A system for removing H₂S from methane which comprises:
(a) providing a reactor cartridge having an outer housing which contains a gas, water inlets, outlets at opposite ends, a source of microorganisms contained within said housing which are selected to degrade H₂S;
(b) passing a stream of methane which contains H₂S through said gas inlet and through said microorganism contained in said housing whereby said H₂S is degraded; and
(c) passing said treated methane through said gas outlet for storage or to a source of use.

16. The system of claim 14 in which the reactor is cylindrical or generally cubic in shape and positioned vertically in operation whereby the methane to be treated is introduced into the reactor from the top through a vertical tube which extends to the bottom which allows the H₂S containing methane to flow upwardly through the microorganisms in the cartridge and out of the gas outlet at the top end of said cartridge.

17. A system for removing H₂S from methane generated by animal waste which comprises:
(a) providing a source of animal waste which generate a biogas which contains a major portion of methane containing a relatively small amount of hydrogen sulfide (H₂S);
(b) providing a reactor cartridge having an outer housing which contains a gas inlet, water inlet and gas outlet at one end, and a source of microorganisms contained within said housing which are selected to degrade H₂S;
(c) passing a stream of said methane containing said H₂S through said gas inlet and through said microorganism contained in said housing whereby said H₂S is degraded;
(d) introducing a downward flow of flushable water, and
(e) passing said treated methane through said gas outlet for storage or to a source of use.

18. The system of claim 14 in which the reactor is cylindrical or generally cubic in shape and positioned vertically in operation whereby the methane to be treated is introduced into the reactor from the top through a vertical tube which allows the H₂S containing methane to flow upwardly through the microorganisms in the cartridge and out of the gas outlet at the top end of said cartridge.

19. The system of claim 17 in which said animal waste comprises cow manure.
